(19) United States
(12) Patent Application Publication
(10) Pub. No.: US 2024/0036297 A1
(43) Pub. Date: Feb. 1, 2024

(54) OPTICAL AXIS COMPENSATOR FOR MANUALLY GUIDED VISUAL EXAMINATION WITH VISION AID

(71) Applicant: Welch Allyn, Inc., Skaneateles Falls, NY (US)

(72) Inventor: Lei Guo, Manlius, NY (US)

(21) Appl. No.: 18/227,251

(22) Filed: Jul. 27, 2023

Related U.S. Application Data
(60) Provisional application No. 63/393,790, filed on Jul. 29, 2022.

(51) Int. Cl. G02B 17/00

(52) U.S. Cl. CPC G02B 17/008; A61B 3/12

(57) ABSTRACT

Apparatuses provide an optical axis compensator configured to provide a smartphone camera with an offset optical axis dislocated from the camera image sensor. A smartphone whose camera has an offset optical axis can be mounted to a manually guided vision aid, such that, while the manually guided vision aid provides a viewing lens to adapt the smartphone camera for photography at non-native focal lengths, the image sensor of the smartphone camera need not be aligned with the viewing lens, providing greater degrees of freedom in mounting the smartphone to the manually guided vision aid, and greater ease in handling the manually guided vision aid with the smartphone mounted thereto, and in using the smartphone for photography while mounted to the manually guided vision aid.

### CROSS REFERENCE TO RELATED APPLICATION

This application is a Nonprovisional of, and claims priority to, U.S. Provisional Patent Application No. 63/393,790, filed Jul. 29, 2022, the entire disclosure of which is incorporated herein by reference.

### BACKGROUND

In various medical fields, clinicians diagnose health and diseases of patients by operating vision aid instruments to examine parts of a patient's body. By way of example, during eye examinations by clinicians, ophthalmoscopes allow a clinician to examine the fundus, the rear interior of the eye, directly. During ear examinations by clinicians, otoscopes allow a clinician to examine the ear canal and eardrum. During examination of skin lesions, dermatoscopes allow a clinician to examine the surface and subsurface of the skin.

Ophthalmoscopes, otoscopes, dermatoscopes, and such vision aid instruments are compact, portable optical instruments which can be handheld or otherwise manually guided. An ophthalmoscope includes a lens which is held by a clinician, whether gripped directly or gripped by an attached member or device, between the patient and the clinician. The ophthalmoscope lens gathers or condenses light exiting a patient's pupil, presenting an image which the clinician can view.

The use of ophthalmoscopes, as well as other such manually guided vision aid instruments, presents substantial physical dexterity challenges, as the clinician must manually align the lens with the very small opening provided by the pupil of the human eye, which is subject to constant, involuntary micro-movements. The manual guidance of such vision aid instruments, whether by a handle or by an attached member device of varying ease of handling, constantly tests the handling ability of clinicians.

Likewise, in general-purpose applications for instrument-based visual examination of objects such as telescopy and microscopy, vision aids can be placed between an eyepiece of an optical instrument and an operator to facilitate viewing of images. Ease of handling can also facilitate the use of vision aids in these instrument-based applications.

Thus, there is a need to allow manually guided vision aids to be used more conveniently in clinical examination and other instrument-based visual examination.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items or features.

FIG. 1 illustrates a smartphone.

FIGS. 2A through 2D illustrate mountings of a smartphone to a manually guided vision aid.

FIG. 3A illustrates possible dislocations of an optical axis of a smartphone camera from the camera image sensor.

FIGS. 3B and 3C illustrate examples of a housing of an optical axis compensator according to example embodiments of the present disclosure.

FIGS. 4A and 4B illustrate examples of an optical axis compensator implemented as a waveguide according to example embodiments of the present disclosure.

FIGS. 5A and 5B illustrate examples of an optical axis compensator implemented as a lens relay system according to example embodiments of the present disclosure.

FIG. 6 illustrates an example of an optical axis compensator as a mirror relay system according to example embodiments of the present disclosure.

FIGS. 7A and 7B illustrate a smartphone manually guided a manually guided vision aid in a balanced fashion according to example embodiments of the present disclosure.

FIG. 8 illustrates another possible dislocation of an optical axis of a smartphone camera from the camera image sensor.

FIGS. 9A and 9B illustrate an example of an optical axis compensator as a fiber optic bundle according to example embodiments of the present disclosure.

### DETAILED DESCRIPTION

Apparatuses discussed herein are directed to implementing photonic components, and more specifically providing an optical axis compensator configured to provide a smartphone camera with an offset optical axis dislocated from the camera image sensor. A smartphone whose camera has an offset optical axis can be manually guided alongside a manually guided vision aid, such that, while the vision aid provides a lens to adapt the smartphone camera for visual examination, and, optionally, photography at both native and non-native focal lengths, the smartphone camera and the vision aid are manually guided in sync, and the image sensor of the smartphone camera need not be aligned with the vision aid lens, providing greater degrees of freedom in mounting the smartphone to the manually guided vision aid, and greater ease in handling the manually guided vision aid alongside the smartphone, and in using the smartphone for visual examination, and, optionally, photography by manually guiding the vision aid.

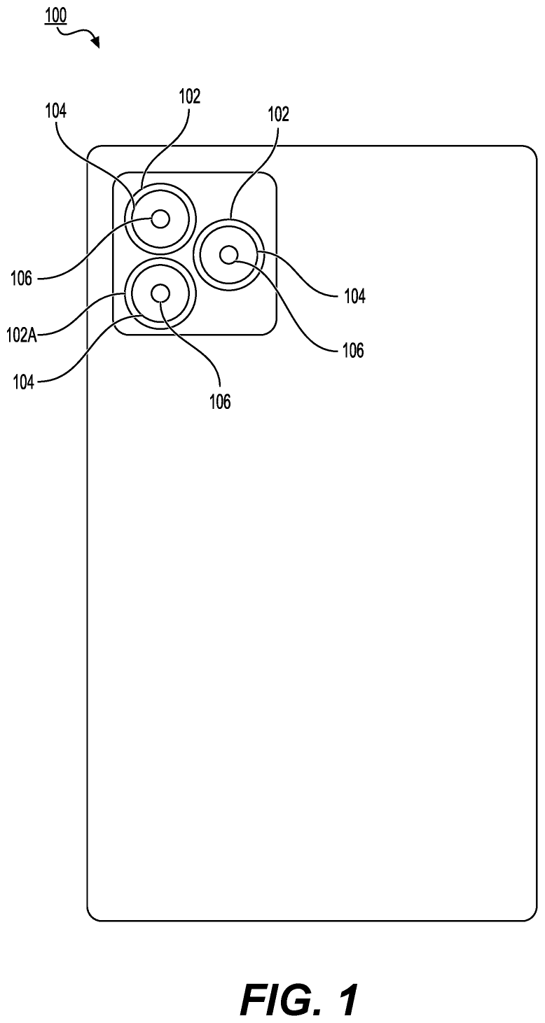

FIG. 1 illustrates a smartphone 100. The smartphone 100 includes a camera 102, which further includes a prime lens 104 (i.e., a photographic lens of fixed focal length), an aperture 106 in front of the prime lens 104, and an image sensor (not illustrated) behind the aperture 106. With few exceptions, smartphone models generally include only prime lens of fixed focal length, and do not include zoom lens having mechanical assemblies allowing variation of focal length except in a small number of models. Furthermore, the aperture 106 is generally also fixed in size in order to minimize camera form factor by eliminating mechanical mass.

In smartphones, a same camera 102 can include multiple prime lenses 104, to simulate the effects of a mechanical zoom lens by switching between the multiple prime lenses 104. Furthermore, a same smartphone 100 can include multiple cameras 102, such as a wide camera module, an ultra-wide camera module, a telephoto camera module, a macro camera module, a depth camera module, and the like.

In a same smartphone 100 with multiple cameras 102, prime lenses 104 of different cameras can have different focal lengths, and therefore different cameras 102 of a same smartphone 100 can capture images at different highest focal lengths (where higher focal lengths permit capture of images with larger magnification). Therefore, among multiple cameras 102 of a same smartphone 100, one camera among these can have a highest focal length (subsequently referred to as a “highest focal length camera 102A” of a smartphone, for brevity). By way of example, certain smartphone models include three cameras 102, and one camera among these three can be a highest focal length camera 102A, where the highest focal length camera 102A has at least one prime lens having a higher focal length than each other camera of the same smartphone.

Thus, prime lenses of a camera can encompass a continuous range of focal lengths; though prime lenses of smartphone cameras cannot mechanically zoom to intervening focal lengths between their fixed focal lengths, smartphones are further configured by computer-executable instructions stored on one or more memories to perform digital zoom post-processing, by one or more processors, on captured images to simulate the intervening focal lengths between the fixed, discrete focal lengths. Thus, for the purpose of the present disclosure, such a continuous range of focal lengths of any individual camera is referred to as a “native focal length range” including any discrete “native focal lengths,” and ranges between them achieved by post-processing, for brevity. Focal lengths outside the native focal length range of an individual camera are referred to as “non-native focal lengths,” for brevity.

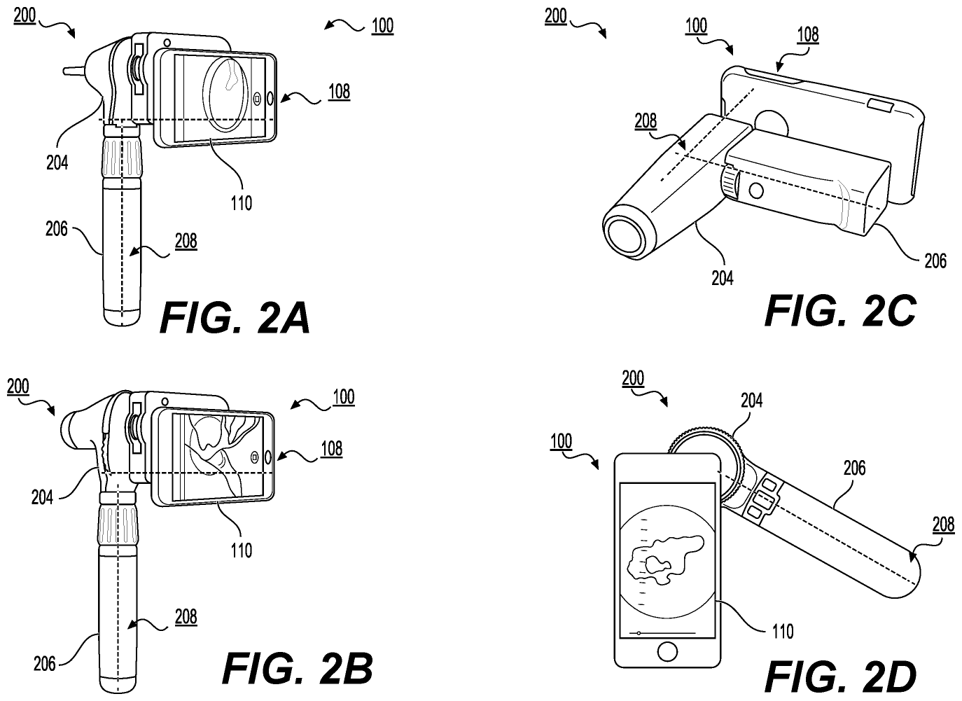

FIGS. 2A through 2D illustrate couplings of a smartphone 100 to a manually guided vision aid 200. The manually guided vision aid 200 can be any instrument where a viewing lens (not illustrated) is set internally within a head 204, which may or may not be further attached to a handle 206. Depending on the nature of the instrument, the instrument can include multiple viewing lenses having various diopter values among various ranges of diopter values. Diopter values describe the optical power of a lens to converge light, thereby compensating for refractive error in an eye of a patient (which can result in light exiting the patient's pupil converging on an unclear image), or refractive error in an eye of a clinician (which can result in the clinician failing to clearly see an image converged from an eye of the patient).

By way of example, in the event that the manually guided vision aid 200 is an ophthalmoscope, it can include multiple viewing lenses having respective diopter values ranging from 1 diopter to 10 diopters (and can further include additional lens having negative diopter values, without limitation). In the event that the manually guided vision aid 200 is an otoscope, a dermatoscope, and other similar instruments, it can likewise include one or more viewing lenses; rather than diopter values, viewing lenses of an otoscope or a dermatoscope generally have focal length specified. A viewing lens of an otoscope can have a focal length of approximately 50 mm, and a viewing lens of a dermatoscope can have a focal length of approximately 25 mm, by way of example.

It should be understood that, while a viewing lens according to example embodiments of the present disclosure can magnify images, magnification of a viewing lens is not relevant to understanding the present disclosure. Thus, by way of example, a viewing lens according to example embodiments of the present disclosure can have angular magnification of approximately 1×, or can have any other arbitrary magnification.

A viewing lens can be set within the head 204 of the manually guided vision aid 200. Furthermore, aspheric lenses found in ophthalmoscopes can have a diopter value of 20 diopters, 28 diopters, 60 diopters, 78 diopters, 90 diopters, and the like; and any of these aspheric lenses can be set within the manually guided vision aid 200, increasing optical power to converge light through one or more viewing lenses.

Thus, as illustrated in FIGS. 2A through 2D, a smartphone 100 can be coupled to a manually guided vision aid 200 such that a prime lens 104 and an aperture 106 of any one camera 102 of the smartphone 100 are aligned with a viewing lens. By interposing this viewing lens in an optical axis 108 (illustrated by a broken line orthogonal to the smartphone body) of the camera 102, where the optical axis 108 describes a path by which light is acquired by an image sensor of the camera 102 after passing through the prime lens 104 and the aperture 106, the viewing lens converges light into images which can be captured by the camera 102, even if the light originates from beyond a native focal length range of the camera 102. In the event that the viewing lens has a diopter value comparable to an aspheric lens found in fundus cameras, the viewing lens can configure the image sensor of the camera 102 to capture images of the rear interior of the human eye by acquiring light on an optical path through the pupil of the human eye while focusing on the fundus of the human eye.

It should be understood that a manually guided vision aid 200 such as an ophthalmoscope having multiple viewing lenses can be configured to mechanically swap between the multiple viewing lenses, to vary a focal length which is interposed in the optical axis 108.

A person who is accustomed to handling any kind of manually guided vision aid 200 to visually examine subjects through a lens can, in a similar manner, handle any kind of manually guided vision aid 200 having a smartphone 100 coupled thereto (whether the vision aid includes a handle 206 or not; in the event that the visual aid does not include a handle, the person can handle the manually guided vision aid 200 by handling the smartphone 100, rather than handling the smartphone 100 by the manually guided vision aid 200) in the course of visually examining subjects, to concurrently operate a camera 102 of the smartphone 100 to capture images of an eye (in the event that the manually guided vision aid 200 is an ophthalmoscope); capture images of an ear (in the event that the manually guided vision aid 200 is an otoscope); capture images of skin (in the event that the manually guided vision aid 200 is a dermatoscope); capture images through an optical instrument (such as a microscope, telescope, or borescope); or capture images of any other subject being examined.

However, cameras of various smartphone models are nearly universally located at a corner of the smartphone body, or otherwise located at one lengthwise end of the smartphone body, for various reasons determined by smartphone manufacturers. As a consequence of such design trends, as illustrated in FIGS. 2A through 2D, the mass of the smartphone 100 cannot be balanced with respect to the manually guided vision aid 200 while coupled thereto. In order to align the viewing lens with the optical axis 108 of the camera 102, a lengthwise end of the body of the smartphone 100 can be substantially aligned with a central axis 208 of the manually guided vision aid 200 (illustrated by a broken line parallel to the instrument body), as illustrated in FIGS. 2A and 2B. Alternatively, a widthwise side of the body of the smartphone 100 can be substantially aligned with the central axis 208 of the manually guided vision aid 200, as illustrated in FIG. 2C. Alternatively, a corner of the body of the smartphone 100 can be substantially aligned with the central axis 208 of the manually guided vision aid 200, as illustrated in FIG. 2D.

In each of these cases, the mass of the smartphone 100 is substantially off-center and imbalanced while coupled to the manually guided vision aid 200, rendering the manually guided vision aid 200 awkward to manually handle. As modern smartphone models increase in size and mass, this off-centering and unbalancing effect becomes further exacerbated.

Additionally, the optical axis 108 of the camera 102, being at a corner of the smartphone body or otherwise at a lengthwise end of the smartphone body, is also offset from the on-screen viewfinder 110 of graphical camera interfaces displayed on a screen of the smartphone 100, which, as illustrated in FIGS. 2A, 2B, and 2D, is usually substantially centered over the body of the smartphone 100. During operation of a camera 102 of a smartphone 100 at native focal length ranges, the offset of the optical axis 108 from the viewfinder 110 is generally not noticed. However, during operation of a camera 102 of a smartphone 100 at non-native focal lengths enabled by a viewing lens, the offset of the optical axis 108 from the viewfinder 110 is readily noticed due to the greatly reduced field of view of the camera 102 causing the offset to become much more pronounced, resulting in substantial disorientation to persons handling the manually guided vision aid 200 with the smartphone 100 coupled thereto.

Therefore, example embodiments of the present disclosure provide an optical axis compensator configured to provide a smartphone camera with an offset optical axis dislocated from the camera image sensor.

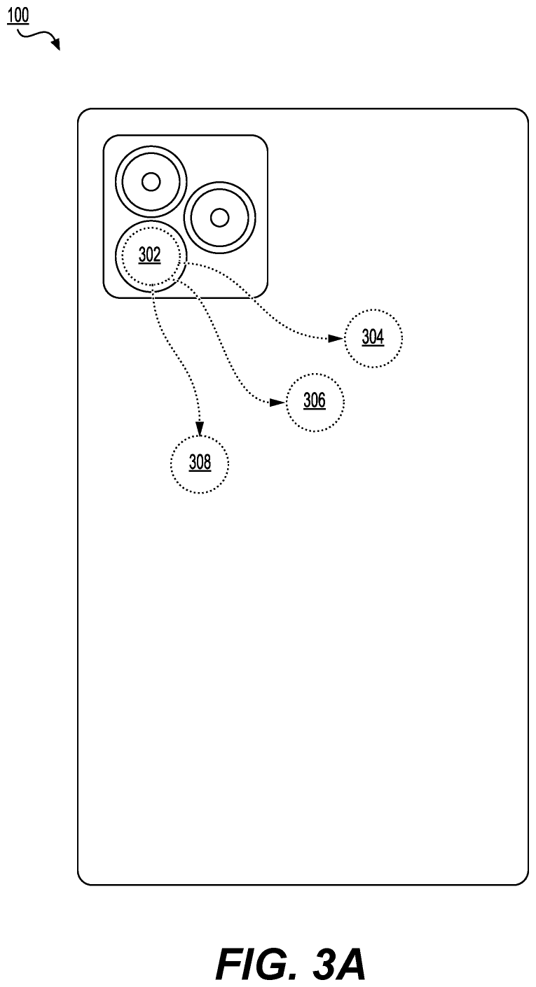

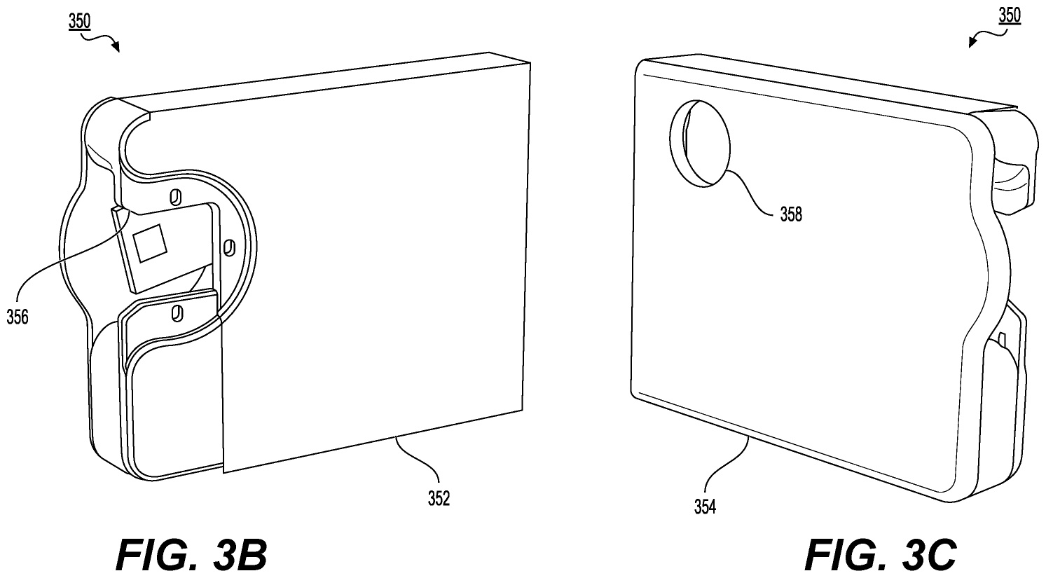

FIG. 3A illustrates possible dislocations of an optical axis of a smartphone camera from the camera image sensor. Given an image sensor located at a position 302 on a smartphone body, thus an original optical axis of a smartphone camera also being located at position 302, example embodiments of the present disclosure permit an offset optical axis to be located at position 304, at position 306, and position 308, or at any other arbitrary position on the smartphone body which is generally closer to a center of the smartphone body than to extremities of the smartphone body.

According to example embodiments of the present disclosure, an optical axis compensator can be implemented as any optical element, optical assembly, photonic element, or photonic assembly in accordance with the following examples, without limitation thereto.

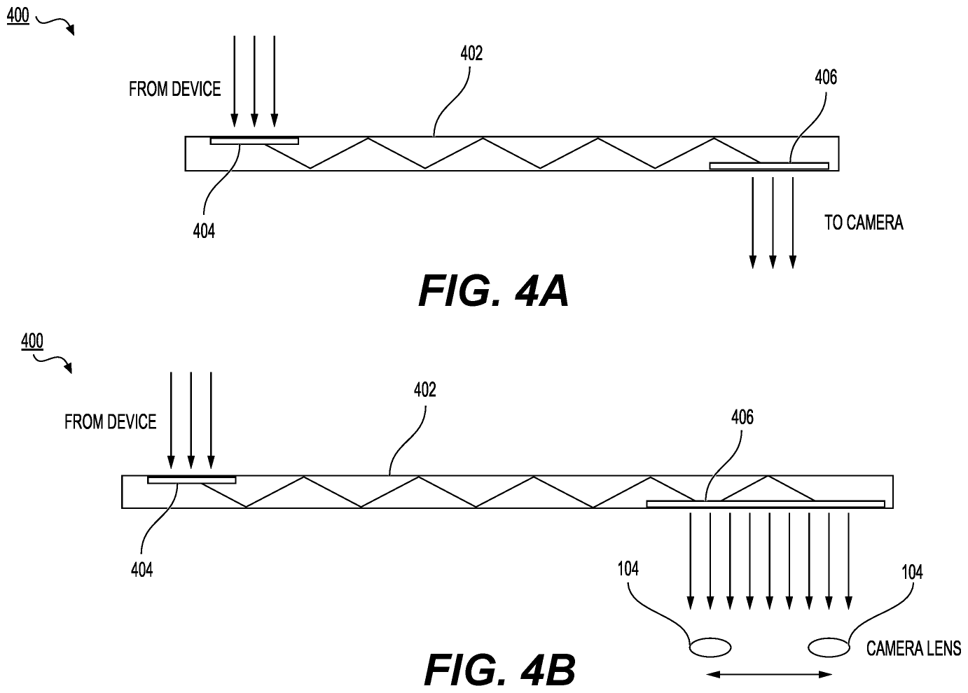

FIGS. 3B and 3C illustrate examples of a housing 350 of an optical axis compensator according to example embodiments of the present disclosure. The housing 350 encompasses any optical axis compensator 400, 500, or 600 as shall be subsequently described with reference to FIG. 4A, 4B, 5A, 5B, or 6. The housing 350 further has a first face 352 which couples adjacent to a manually guided vision aid 200, and a second face 354 which couples adjacent to a smartphone 100. The first face 352 can couple adjacent to any manually guided vision aid 200 by any physically mating configuration, mechanical bracket, mechanical clamp, removable adhesive, and the like. The second face 354 can couple adjacent to any smartphone 100 by any physically mating configuration, mechanical bracket (such as a mechanical bracket 700 as shall be described subsequently), mechanical clamp, removable adhesive, and the like.

The first face 352 further includes a first viewport 356, and the second face further includes a second viewport 358. The first viewport 356 exposes the in-coupler 404, the first mirror 502, or the incident mirror 602 to incident light as described subsequently, and the second viewport 358 exposes the out-coupler 406, the second mirror 504, or the exit mirror 604 as described subsequently.

FIGS. 4A and 4B illustrate examples of an optical axis compensator 400 implemented as a waveguide according to example embodiments of the present disclosure.

The optical axis compensator 400 can be a holographic waveguide having an input coupler (“in-coupler”) and an output coupler (“out-coupler”). A holographic waveguide includes a substrate medium which traps and guides light within by total internal reflection, where the in-coupler couples light from air into the glass, and the out-coupler couples light from within the glass back into air. The in-coupler and the out-coupler are each transmission holographic optical elements (“HOEs”) fabricated from photopolymer resin, each HOE recording a transmission holographic grating.

As illustrated in FIGS. 4A-4B, the optical axis compensator 400 includes a substrate medium 402 (such as glass), fabricated having two substantially planar faces on opposing sides, having an in-coupler 404 and an out-coupler 406 on opposite faces of the total internal reflection of the substrate medium 402. Light incident on the in-coupler 404 is coupled from air into the substrate medium 402, wherein, by total internal reflection, a holographic image is transmitted through the substrate medium 402, reflected internally by the two faces of the substrate, which guide the holographic image to the out-coupler 406, where the holographic image is coupled from the glass back into air.

By placing such an optical axis compensator 400 adjacent to the smartphone body as illustrated in FIGS. 3A-3C, and aligning the out-coupler 406 with the position 302 where the image sensor is located, the optical axis of a smartphone camera can be dislocated to any of positions 304, 306, or 308 by positioning the in-coupler 404 at those respective positions, and can be dislocated to any other arbitrary position by positioning the in-coupler 404 at that respective position. Thus, by transmitting an image through the out-coupler 406 to the image sensor of the smartphone camera, the optical axis compensator 400 provides the smartphone camera with an offset optical axis which is no longer aligned with the image sensor of the camera.

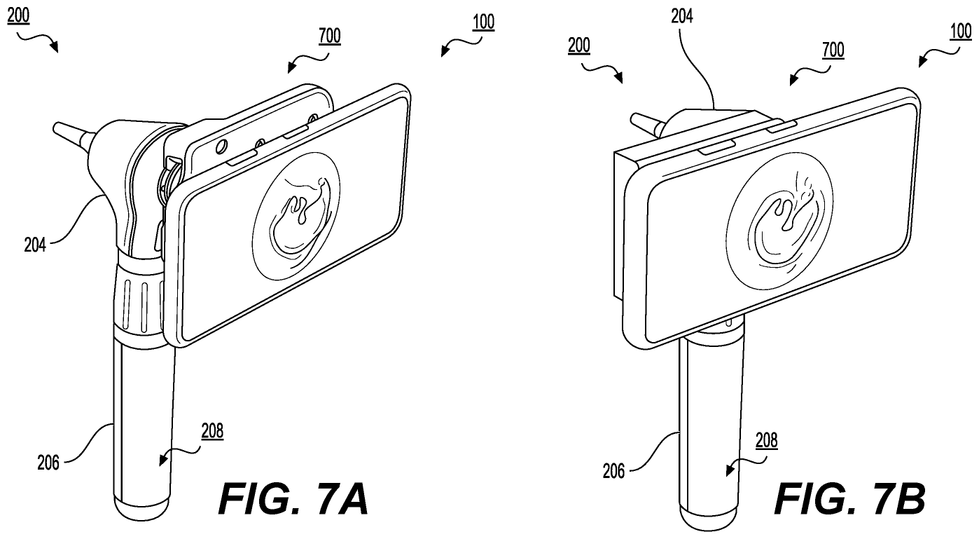

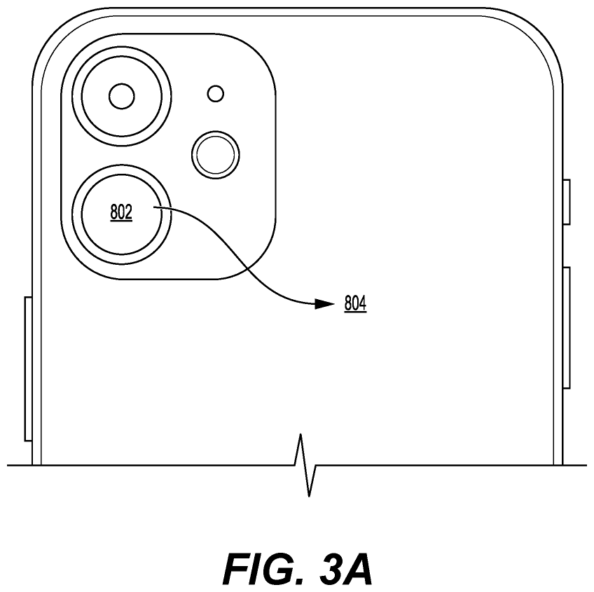

A housing 350 of the optical axis compensator 400 (e.g., a housing that encloses the optical axis compensator) can be coupled to a manually guided vision aid and furthermore coupled to a smartphone, such that the optical axis compensator 400 is adjacent to the smartphone in a fashion as described above when the smartphone is coupled to the housing 350. In this manner, the smartphone can be manually guided alongside a manually guided vision aid in a balanced fashion as subsequently illustrated in FIGS. 7A and 7B.

Furthermore, as illustrated in FIG. 4B, the out-coupler 406 can be substantially larger than a prime lens 104 of a camera 102 of a smartphone 100. Depending on configurations of different smartphone models, an out-coupler 406 may encompass a substantially larger area, so as to enable alignment with differently positioned cameras 102 and prime lens 104, as these elements are fixed and cannot be repositioned. It should be understood that, by the optical characteristics of an out-coupler of a holographic waveguide, a substantially same image can be captured by the prime lens 104 regardless of the positioning of the prime lens 104 relative to the out-coupler 406.

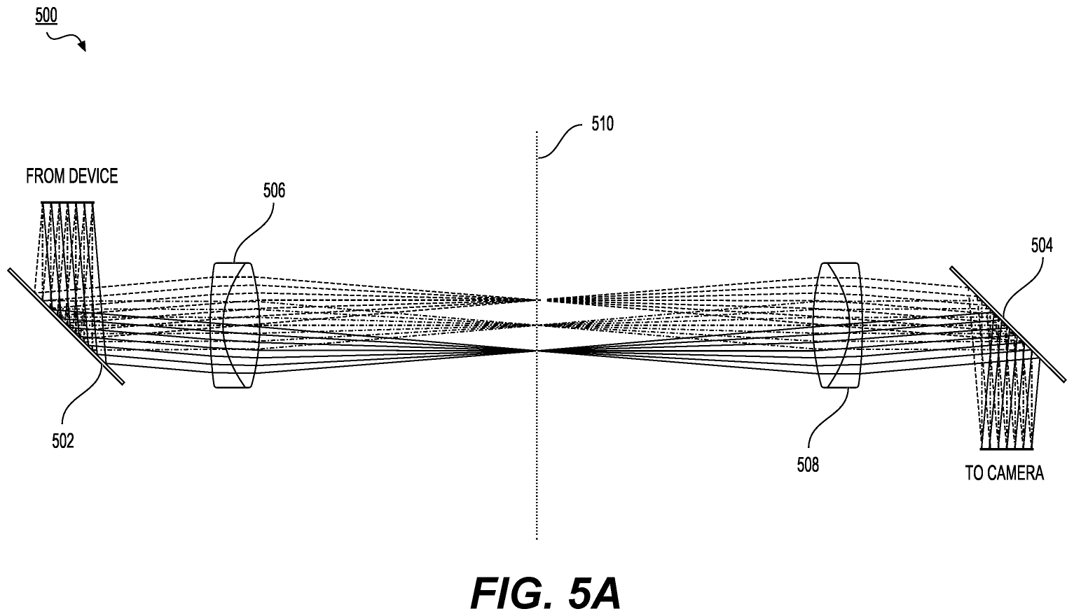

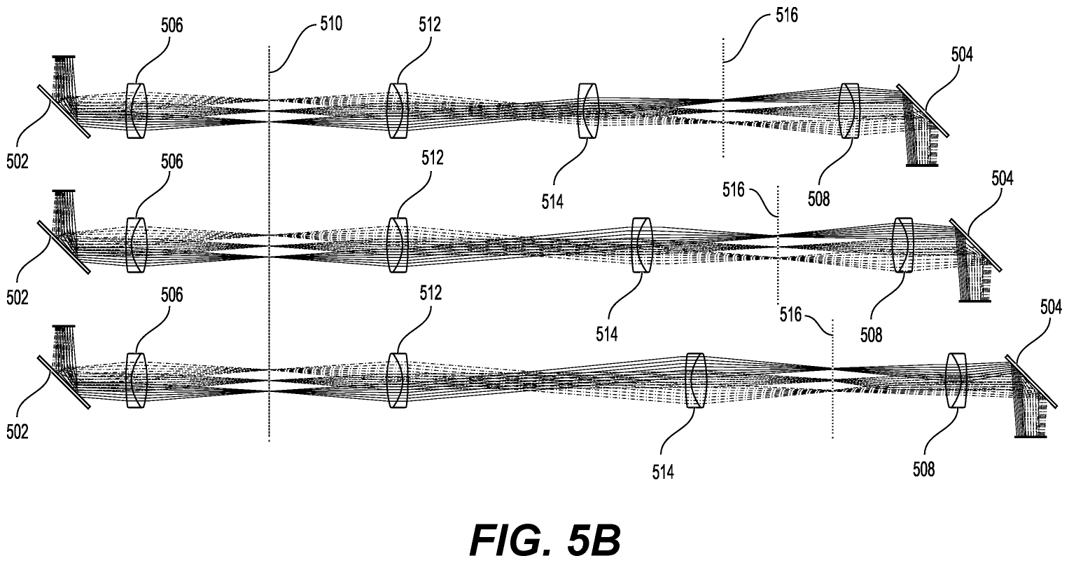

FIGS. 5A and 5B illustrate examples of an optical axis compensator 500 implemented as a lens relay system according to example embodiments of the present disclosure.

The optical axis compensator 500 can include a first mirror 502 and a second mirror 504, with a first relay lens 506 and a second relay lens 508 situated in a lateral axis between the first mirror 502 and the second mirror 504. The first mirror 502 is oriented at an angle from a direction of incident light from a viewing lens coupled to a manually guided vision aid as described above, while the second mirror 504 is positioned laterally across from, and oriented at an angle parallel to, the first mirror 502. Light producing an image is incident on, and is reflected by, the first mirror 502 at an angle. Between the first mirror 502 and the second mirror 504, the first relay lens 506 focuses a reflected image from the first mirror 502 onto an intermediate image plane 510 between the first relay lens 506 and the second relay lens 508. Past the intermediate image plane 510, the focused image is focused by the second relay lens 508 onto the second mirror 504, which reflects the image out of the lens relay system at an angle substantially parallel to the direction of incident light.

By placing such an optical axis compensator 500 adjacent to the smartphone body as illustrated in FIGS. 3A-3C, and aligning the second mirror 504 with the position 302 where the image sensor is located, the optical axis of a smartphone camera can be dislocated to any of positions 304, 306, or 308 by positioning the first mirror 502 at those respective positions (as long as the first relay lens 506 and the second relay lens 508 remain positioned between the first mirror 502 and the second mirror 504, and each of these elements remains in alignment in the lateral axis as illustrated in FIGS. 5A-5B), and can be dislocated to any other arbitrary position by positioning the first mirror 502 at that respective position. Thus, by reflecting a relayed image from the second mirror 504 to the image sensor of the smartphone camera, the optical axis compensator 500 provides the smartphone camera with an offset optical axis which is no longer aligned with the image sensor of the camera.

Furthermore, the first relay lens 506 and the second relay lens 508 have different focal length, so the system magnification is not necessarily 1. In this case, the magnification is fixed. Furthermore, Either the first relay lens 506 or the second relay lens 508 or both are composed of zoomable optics, so that the system magnification can vary depending on a person's need to capture magnified or demagnified images using the smartphone camera.

Furthermore, as illustrated in FIG. 5B, a third relay lens 512 and a fourth relay lens 514 can be added in alignment to the lateral axis, the third relay lens 512 pairing with the first relay lens 506 to transmit the focused image from the intermediate image plane 510 to the fourth relay lens 514, and the fourth relay lens 514 focuses the transmitted image onto a second intermediate image plane 516, which is re-focused by the second relay lens 508 as described above. The relay lens system can be further extended in this fashion with the addition of further pairs of relay lens. The extension of the relay lens system enables the optical axis compensator 500 to dislocate the optical axis to arbitrary distances. Furthermore, the distance between the third relay lens 512 and the fourth relay lens 514 can vary so that the optical axis compensator 500 has variable length.

A housing 350 of the optical axis compensator 500 can be coupled to a manually guided vision aid and furthermore coupled to a smartphone, such that the optical axis compensator 500 is adjacent to the smartphone in a fashion as described above when the smartphone is couple to the housing 350. In this manner, the smartphone can be manually guided alongside a manually guided vision aid in a balanced fashion as subsequently illustrated in FIGS. 7A and 7B.

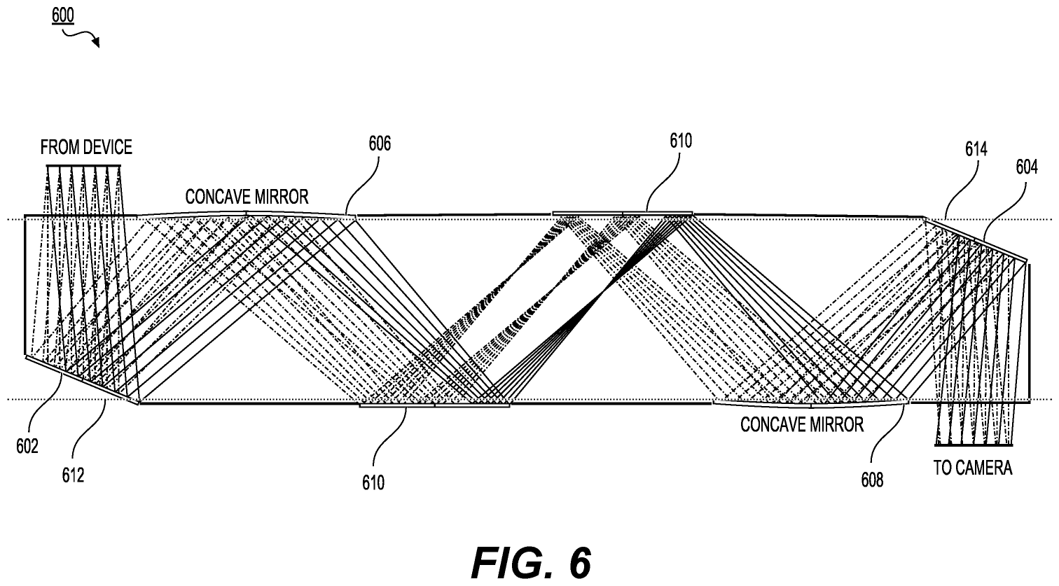

FIG. 6 illustrates an example of an optical axis compensator 600 as a mirror relay system according to example embodiments of the present disclosure.

The optical axis compensator 600 can include at least an incident mirror 602, an exit mirror 604, a first concave mirror 606, a second concave mirror 608, and any number of further non-concave mirrors. The incident mirror 602 is positioned at a first lateral axis 612 and oriented at an angle from a direction of incident light from a viewing lens coupled to a manually guided vision aid as described above, while the exit mirror 604 is positioned at a second lateral axis 614 and oriented at a second parallel to the incident mirror 602. The first lateral axis 612 is further from the viewing lens than the second lateral axis 614.

Light producing an image is incident on, and is reflected by, the incident mirror 602 at an angle. The first concave mirror 606 is positioned along the second lateral axis 614 such that it reflects the reflected image from the incident mirror 602. A number of non-concave mirrors are positioned alternatingly along the first lateral axis 612 and second lateral axis 614 such that they reflect the reflected image, in series, to the second concave mirror 608, which is positioned along the first lateral axis 612 such that it reflects the reflected image to the exit mirror 604. The exit mirror 604 reflects the image out of the mirror relay system at an angle substantially parallel to the direction of incident light. The design can be extended by adding extra mirrors for longer distance so that the incident light and exiting light can be further apart. In an alternative design not shown, the incident mirror 602, the second concave mirror 608, and the non-concave mirror 610 may not be positioned along the first lateral axis 612. Also the exit mirror 604, the first concave mirror 606 and the non-concave mirror 610 may not be positioned along the second lateral axis 614. The design can be varied by mix and match from flat, concave and convex mirrors. In another design, freeform surfaces could be used to replace each mirror and furthermore, all surfaces are integrated together so it is not possible to identify each individual mirror.

Each of the above-described mirrors can each be individually held in place, or can be integrated into a molded light-transmitting substrate.

By placing such an optical axis compensator 600 adjacent to the smartphone body as illustrated in FIGS. 3A-3C, and aligning the exit mirror 604 with the position 302 where the image sensor is located, the optical axis of a smartphone camera can be dislocated to any of positions 304, 306, or 308 by positioning the incident mirror 602 at those respective positions, and can be dislocated to any other arbitrary position by positioning the incident mirror 602 at that respective position. Thus, by reflecting a relayed image from the exit mirror 604 to the image sensor of the smartphone camera, the optical axis compensator 600 provides the smartphone camera with an offset optical axis which is no longer aligned with the image sensor of the camera.

A housing 350 of the optical axis compensator 600 can be coupled to a manually guided vision aid and further coupled to a smartphone, such that the optical axis compensator 600 is adjacent to the smartphone in a fashion as described above when the smartphone is coupled to the housing 350. In this manner, the smartphone can be manually guided alongside a manually guided vision aid in a balanced fashion as subsequently illustrated in FIGS. 7A and 7B.

FIGS. 7A and 7B illustrate a smartphone 100 manually guided alongside a manually guided vision aid 200 in a balanced fashion according to example embodiments of the present disclosure. By way of example, a mechanical bracket 700 is mounted to the head 204 of the manually guided vision aid 200, on a viewing side of the lens.

Within the mechanical bracket 700 as illustrated in FIG. 7B, it should be understood that an optical axis compensator according to example embodiments of the present disclosure, such as, without limitation thereto, the optical axis compensator 400 of FIGS. 4A-4B, the optical axis compensator 500 of FIGS. 5A-5B, or the optical axis compensator 600 of FIG. 6, is mounted within the mechanical bracket 700, with a first viewport 356 exposing the in-coupler 404, exposing the first mirror 502, exposing the incident mirror 602, or otherwise exposing a point at which light producing an image enters the optical axis compensator (each of these subsequently referenced as a “light incident point”) aligned with the viewing lens.

In both FIG. 7A and FIG. 7B, a smartphone 100 is coupled to the mechanical bracket 700. However, in FIG. 7A, without the optical axis compensator being mounted in the mechanical bracket 700 first, a camera 102 of the smartphone 100 is aligned with the viewing lens, resulting in the mass of the smartphone 100 being substantially off-center and imbalanced. In contrast, in FIG. 7B, with the optical axis compensator being mounted in the mechanical bracket 700, the camera 102 of the smartphone 100 is not aligned with the viewing lens, but rather aligned with a location of the out-coupler 406, a location of the second mirror 504, a location of the exit mirror 604, or otherwise a point at which light producing an image exits from the optical axis compensator (each of these subsequently referenced as a “light exit point”), resulting in the mass of the smartphone 100 being balanced with respect to the manually guided vision aid 200.

Furthermore, it should be understood that the clearance required for the thickness of the optical axis compensator to fit between the mechanical bracket 700 and the smartphone 100 also provides clearance for added thickness of a camera 102 protruding from the back of the smartphone 100. Due to cameras of smartphone models having increasingly greater thickness to fit more complex assemblies of prime lenses with longer focal lengths, such additional clearance of approximately 1-2 millimeters can be beneficial for permitting coupling of present and future smartphone models.

FIG. 8 illustrates another possible dislocation of an optical axis of a smartphone camera from the camera image sensor. Given an image sensor located at a position 802 on a smartphone body, thus an original optical axis of a smartphone camera also being located at position 802, example embodiments of the present disclosure permit an offset optical axis to be located at position 804, or at any other arbitrary position on the smartphone body or even beyond the smartphone body which is generally closer to an extremity of the smartphone body than to a center of the smartphone body.

Such dislocation of the optical axis is possible according to any of the above example embodiments, without limitation thereto. Such dislocation of the optical axis can be configured on one or more cameras of the same single smartphone so that the offset optical axes of both or all cameras can be brought into lateral adjacency, enabling the two or more cameras to be operated in conjunction (whether manually or by automated synchronization using installed software) to perform stereoscopic photography of a overlapped field of view from two or more converging or offset optical axes. Without dislocating optical axes, such stereoscopic photography would be greatly challenging due to the short inter-camera distance. This concept can be extended to more than one smartphone which was used in conjunction. Furthermore, there are applications beyond stereoscopic photography in which the dislocated camera axis can be diverging into different directions with or without overlapped view to extend the overall field of view. using two cameras of two smartphones manually guided alongside one or to two manually guided vision aids, since the two smartphones are rotationally symmetrical but not axially symmetrical, preventing both cameras from being brought into lateral adjacency with both smartphones being in the same orientation.

Consequently, an optical axis compensator according to example embodiments of the present disclosure configures smartphones to be manually guided alongside vision aids without off-centering or unbalancing the mass of the smartphone, facilitating handling of the manually guided vision aid by persons alongside a smartphone, and facilitating operating of both the manually guided vision aid and the smartphone for imaging. Moreover, the optical axis compensator can substantially eliminate the offset of the optical axis of the smartphone camera from the on-screen viewfinder of graphical camera interfaces displayed on a screen of the smartphone, reducing disorientation to persons handling the manually guided vision aid with the smartphone coupled thereto in the course of utilizing dexterity to precisely photograph minute, magnified fields of view.

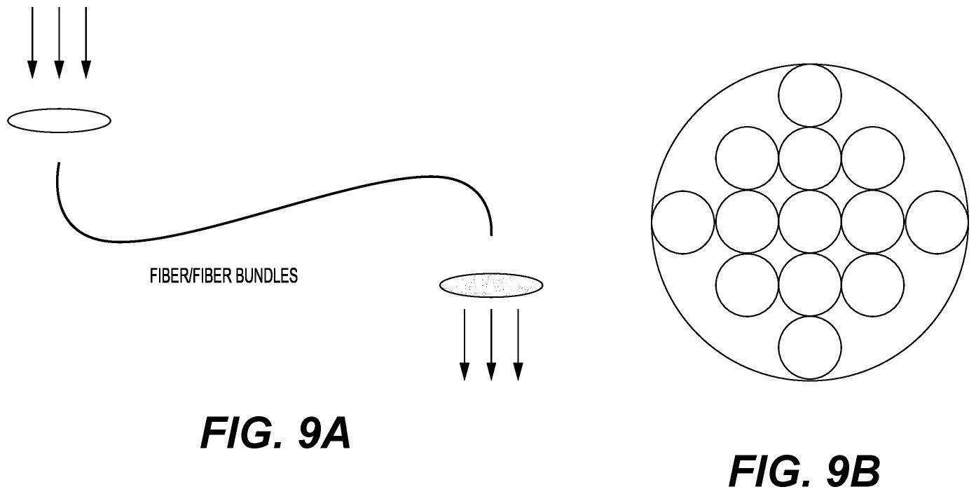

FIGS. 9A and 9B illustrate an example of an optical axis compensator as a fiber optic bundle according to example embodiments of the present disclosure. The light incident point as described above should be understood as a first end of a bundle of fiber optics, and the light exit point as described above should be understood as a second end of the bundle of fiber optics. An in-coupler may or may not be present at the first end, and an out-coupler may or may not be present at the second end. The number of fibers can vary, and a cross-section of each individual fiber or the fiber bundle as a whole may or may not be circular. The optical axis compensator may not be encompassed in a housing, and the housing may merely have the manually guided visual aid and the smartphone coupled thereto.

Although smartphones and camera modules on the back of the smartphones are described, as a general understanding, the concept is not limited to the smartphone and any specific camera module. A camera 102 according to example embodiments of the present disclosure should be understood as encompassing any camera module on smartphones including wide camera, telephoto camera, ultra-wide camera, macro camera and face camera. A smartphone 100 according to example embodiments of the present disclosure should be understood as encompassing any electronic device encompassing at least one camera which is not substantially centered in a mass of the electronic device, including professional camera, point-and-shoot camera and action camera, any photonic detector, such as ambient light sensor, proximity sensor, lidar sensor and biometric sensor, any component that uses photons or electromagnetic waves as the input.

Although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

What is claimed is:

1. A housing, comprising:
a viewport, and
an optical axis compensator enclosed within the housing;
wherein the housing is configured to be coupled to a manually guided vision aid and is configured to be coupled to an electronic device having a mass and including a camera not substantially centered in the mass, such that a light incident point of the optical axis compensator is aligned with a lens of the manually guided vision aid through the viewport, and a light exit point of the optical axis compensator is aligned with the camera of the electronic device.

2. The housing of claim 1, wherein the optical axis compensator comprises a waveguide.

3. The housing of claim 2, wherein the waveguide comprises:
a substrate medium;
an in-coupler; and
an out-coupler;
wherein:
the in-coupler is located at the light incident point; and
the out-coupler is located at the light exit point.

4. The housing of claim 1, wherein the optical axis compensator comprises a lens relay system.

5. The housing of claim 4, wherein the lens relay system comprises:
a first mirror;
a second mirror;
a first relay lens; and
a second relay lens;
wherein:
the first mirror, the first relay lens, the second relay lens, and the second mirror are each aligned in a lateral axis;
the first mirror is located at the light incident point; and
the second mirror is located at the light exit point.

6. The housing of claim 1, wherein the optical axis compensator comprises a mirror relay system.

7. The housing of claim 6, wherein the mirror relay system comprises:
an incident mirror;
an exit mirror;
a first concave mirror; and
a second concave mirror;
wherein:
the incident mirror and the second concave mirror are aligned in a first lateral axis;
the exit mirror and the first concave mirror are aligned in a second lateral axis;
the incident mirror is located at the light incident point; and
the exit mirror is located at the light exit point.

8. An optical axis compensator, comprising:
a first substantially planar face and a second substantially planar face on opposing sides;
a light incident point on the first substantially planar face; and
a light exit point on the second substantially planar face;
wherein the light incident point and the light exit point are substantially offset from each other along an axis orthogonal to the first substantially planar face and the second substantially planar face.

9. The optical axis compensator of claim 8, wherein the optical axis compensator comprises a waveguide.

10. The optical axis compensator of claim 9, wherein the waveguide comprises:
a substrate medium;
an in-coupler; and
an out-coupler;
wherein:
the in-coupler is located at the light incident point; and
the out-coupler is located at the light exit point.

11. The optical axis compensator of claim 8, wherein the optical axis compensator comprises a lens relay system.

12. The optical axis compensator of claim 11, wherein the lens relay system comprises:
a first mirror;
a second mirror;
a first relay lens; and
a second relay lens;
wherein:
the first mirror, the first relay lens, the second relay lens, and the second mirror are each aligned in a lateral axis;
the first mirror is located at the light incident point; and
the second mirror is located at the light exit point.

13. The optical axis compensator of claim 8, wherein the optical axis compensator comprises a mirror relay system.

14. The optical axis compensator of claim 13, wherein the mirror relay system comprises:
an incident mirror;
an exit mirror;
a first concave mirror; and
a second concave mirror;
wherein:
the incident mirror and the second concave mirror are aligned in a first lateral axis;
the exit mirror and the first concave mirror are aligned in a second lateral axis;
the incident mirror is located at the light incident point; and
the exit mirror is located at the light exit point.

15. A manually guided vision aid, comprising:
at least one optical element; and
a housing coupled to the at least one optical element;
wherein:
the housing further comprises a viewport;
an optical axis compensator is mounted within the housing; and
the housing is configured to be coupled to an electronic device having a mass and including a camera not substantially centered in the mass, such that a light incident point of the optical axis compensator is aligned with a lens through the viewport, and a light exit point of the optical axis compensator is aligned with the camera of the electronic device.

16. The manually guided vision aid of claim 15, wherein the optical axis compensator comprises a waveguide.

17. The manually guided vision aid of claim 16, wherein the waveguide comprises:
a substrate medium;
an in-coupler; and
an out-coupler;
wherein:
the in-coupler is located at the light incident point; and
the out-coupler is located at the light exit point.

18. The manually guided vision aid of claim 15, wherein the optical axis compensator comprises a lens relay system.

19. The manually guided vision aid of claim 18, wherein the lens relay system comprises:
a first mirror;
a second mirror;
a first relay lens; and
a second relay lens;
wherein:
the first mirror, the first relay lens, the second relay lens, and the second mirror are each aligned in a lateral axis;
the first mirror is located at the light incident point; and
the second mirror is located at the light exit point.

20. The manually guided vision aid of claim 15, wherein the optical axis compensator comprises a mirror relay system further comprising:
an incident mirror;
an exit mirror;
a first concave mirror; and
a second concave mirror;
wherein:
the incident mirror and the second concave mirror are aligned in a first lateral axis;
the exit mirror and the first concave mirror are aligned in a second lateral axis;
the incident mirror is located at the light incident point; and
the exit mirror is located at the light exit point.
